(19) United States
(12) Patent Application Publication
(10) Pub. No.: US 2025/0161080 A1
(43) Pub. Date: May 22, 2025

(54) PALMAR STRAP ASSEMBLY

(71) Applicant: RCM ENTERPRISE LLC, Olympia, WA (US)

(72) Inventors: Micah Bryant PARKS, Olympia, WA (US); Gabrielle Ona HILGART, Olympia, WA (US); Bradley Arthur CRITTENDEN, Olympia, WA (US); Ana Rosa ACEVEDO, Olympia, WA (US); Rachel Adsit LOWING, Olympia, WA (US)

(21) Appl. No.: 18/953,538

(22) Filed: Nov. 20, 2024

Related U.S. Application Data
(60) Provisional application No. 63/601,567, filed on Nov. 21, 2023.

(51) Int. Cl. A61F 2/78; A61F 2/58

(52) U.S. Cl. CPC A61F 2/78; A61F 2/586; A61F 2002/7862

(57) ABSTRACT

A prosthetic finger assembly includes one or more integrated attachment points for a palmar strap to prevent a backplate bridging and migration. The palmar strap is integrally fixed to a first anchor of a radial metacarpophalangeal pivot, which is configured to connect to the hand of a user, and removably attach to a second anchor of an ulnar metacarpophalangeal pivot. The palmar strap is removably attached to the first anchor and integrally fixed to the second anchor.

### CROSS-REFERENCE TO RELATED PATENTS AND APPLICATIONS

This application incorporates by reference U.S. Provisional Application No. 63/601,567, filed on Nov. 21, 2023, U.S. Pat. No. 9,629,731, published on Apr. 25, 2017, U.S. Pat. No. 9,707,103, published on Jul. 18, 2017, U.S. Pat. No. 9,713,541, published on Jul. 25, 2017, and U.S. Patent Publication No. 2022/0378586, published on Dec. 1, 2022.

### BACKGROUND

Finger amputations are the most common of upper limb amputations and mostly involve single digits. If a person loses finger mobility, finger functionality, or all or a segment of their physical finger, the result is impaired hand performance. Having an amputated or minimally functioning finger (e.g., due to nerve damage, excessive scar tissue, neurological damage or disorders, or other bone or musculature dysfunctionalities) inhibits the person from performing some of the most basic tasks. For example, typing on a computer keyboard or dialing on a telephone becomes significantly more difficult with one or more dysfunctional fingers. These types of tasks require precise actions that only fingers can offer.

Not only do fingers allow for the performance of precise physical actions, but they also provide an increased ability to grip or handle items. While holding an item in the hand, the item's weight is dispersed through a user's fingers. By varying the force, each finger uses on the holder's hand, the holder can manipulate the item in myriad ways. However, if the holder is missing all or even part of a single finger/digit, or if a finger is present but nonfunctioning, this freedom of manipulation and the number of degrees through which the holder can manipulate the item is drastically decreased.

Over the last decade, the options for partial hand prostheses have increased greatly. Partial hand prostheses are available in both passive mechanical and body-powered designs. Passive mechanical designs include passive articulating devices, which generally use a ratchet-like mechanism to sustain high loads of force at various degrees of flexion. These devices are mechanical in appearance and are frequently used by amputees for manual labor or heavy weight-bearing activities. GripLock Finger™ by Naked Prosthetics is an exemplary passive mechanical prosthesis that works as a positionable, locking mechanical finger.

Body-powered partial hand prostheses rely on the motion of one or more proximal joints to control the prosthetic devices and provide grip strength proportional to the amount of input strength the user provides. The MCP Driver™ by Naked Prosthetics is an exemplary body-powered prosthesis that uses motion from an intact metacarpal-phalangeal joint to actuate the prosthetic device.

Although prosthetic technology has improved over the last decade, the number of prosthetic devices abandoned remains relatively high. Some reasons for abandoning prosthetic devices include weight, difficulty of use, discomfort, lack of functionality, and predictability. Thus, providing a lightweight, comfortable, and intuitive prosthetic hand device would be beneficial.

Additionally, although both passive mechanical and body-powered hand prostheses offer numerous benefits to amputees and individuals with impaired limb performance (i.e., improved mobility, increased independence, reduced psychosocial stress), hand prostheses in the prior art suffer from disadvantages of providing adequate means for connecting the device to the wearer. Existing partial hand prostheses may experience backplate bridging and backplate migration. These issues lead to complications in operating the partial hand prosthesis, causing discomfort and misalignment. Additionally, the prosthetic straps and cables used with these devices can become uncomfortable and difficult to operate, requiring ongoing adjustments and repairs. Thus, there is a need for a partial hand prosthesis equipped with both (1) improved connectivity to prevent backplate bridging and migration and (2) simplified, comfortable securing means to avoid discomfort.

The inventors of the present disclosure have identified a novel and non-obvious solution by integrating a partial hand prosthesis with anchor points of an attachable palmar strap. One skilled in the art would not have suggested or been motivated to add a palmar strap to a partial hand prosthesis because it customarily would have presented the drawbacks of increased bulk and lead time, uncomfortable interference with the distal skin crease of the palm, and the presence of fasteners directly engaged with the skin. However, the attachable palmar strap assembly of the present disclosure overcomes the previously observed drawbacks to prevent bridging of the backplate and enhance overall human-prosthetic interaction.

A significant advantage to the disclosed device is that it allows a palmar strap to be applied to a partial hand prosthesis without modifying the backplate design, thus providing a field serviceable suspension improvement. The disclosed solution for a palmar strap attachment advantageously avoids an entire system (e.g., partial hand prosthesis) revision, which can take several weeks to produce. The disclosed palmar strap assembly can effectively be inventoried and shipped on demand.

### SUMMARY

The attachable palmar strap assembly embodiments of the present disclosure are provided to address the issues of backplate bridging and migration and to offer a lightweight, simplified solution that allows the user of a partial hand prosthesis to acquire a natural, fluent experience with the device. At least one attachment or lashing point is added to the outside anchor washers (i.e., radial and ulnar) of the backplate, also called an anchor platform, to which a palmar strap can be connected. The palmar strap attaches to the backplate's most radial and ulnar anchor positions. A one-finger backplate can incorporate a dual-sided attachment anchor washer in embodiments. The palmar strap advantageously prevents bridging of the backplate, and the palmar strap is functionally positionable about the z-axes (i.e., radial MCP and ulnar MCP axes) of the anchor washers so the strap can be angled to sit in the most comfortable or functional position.

In the first aspect of an embodiment for a partial hand prosthesis comprising more than one prosthetic finger, the palmar strap assembly comprises a dorsal anchor platform or backplate having a radial metacarpophalangeal (MCP) pivot and an ulnar MCP pivot. The radial MCP pivot defines a first anchor washer, also called a palmar washer, and the ulnar MCP defines a second anchor washer. The first anchor washer comprises a first attachment point for the first end of the palmar strap, and the second anchor washer comprises a second attachment point for the second end of the palmar strap. In an embodiment, the first end of the palmar strap is fixed to the first anchor washer. In an alternative embodiment, the second end of the palmar strap is fixed to the second anchor washer. The first anchor washer is positionable about a radial MCP axis, and the second anchor washer is positionable about an ulnar MCP axis, wherein the radial MCP and ulnar MCP axes are defined in ventral-dorsal directions.

In an embodiment of the disclosed strap assembly, the first anchor washer comprises a base tab and an upper tab. The base tab preferably comprises a first opening or aperture corresponding to the radial MCP axis. It is configured to receive a first articulation joint and an arcuate slot adapted to receive the first end of the palmar strap. The upper tab preferably comprises a second aperture corresponding to the radial MCP axis and configured to receive the first articulation joint and an aperture or opening adapted to receive the first end of the palmar strap, the opening corresponding to and being aligned with the arcuate slot of the base tab. The upper and base tabs are mounted to the dorsal anchor platform by the first articulation joint and act as a monolithic unit. In an embodiment, the strap assembly further comprises a wrist strap that defines a first and second end, wherein the first end is integrally secured to a proximal, ulnar end of the dorsal anchor platform (or backplate).

In an embodiment of the strap assembly wherein the first end of the palmar strap is fixed to the first attachment point of the first anchor washer, the second end of the palmar strap is preferably removably attached to the second attachment point of the second anchor washer. In an embodiment of the strap assembly wherein the second end of the palmar strap is fixed to the second attachment point of the second anchor washer, the first end of the palmar strap is preferably removably attached to the first attachment point of the first anchor washer.

In a second aspect of the disclosed palmar strap assembly, an embodiment for a prosthetic hand (or prosthetic finger assembly) comprises a backplate and a foam pad mounted directly upon a palmar side of the backplate. The backplate comprises a radial metacarpophalangeal (MCP) pivot and an ulnar MCP pivot; the radial MCP pivot defines a first anchor washer, and the ulnar MCP defines a second anchor washer. The prosthetic hand further comprises a first articulation assembly rotatably coupled between the radial MCP pivot and a first distal coupler and a second articulation assembly rotatably coupled between the ulnar MCP pivot and a second distal coupler. The palmar strap of the device defines a first end and a second end, and the second end is removably attached to the second anchor washer of the ulnar MCP pivot.

In an embodiment wherein the first end is fixed to the first anchor washer of the radial MCP pivot, the second anchor washer defines an integrated lashing point formed by a first opening and a second opening to receive the second end of the palmar strap. A base tab forms the first opening, and an upper tab forms the second opening. In an embodiment, the second anchor washer may define a receiving ring to receive the second end of the palmar strap. In an embodiment, the second anchor washer may define a textile tab to engage with the second end of the palmar strap. The textile tab comprises hook fastener material, and the palmar strap's second end comprises loop fastener material. In an embodiment wherein the second end is fixed to the second anchor washer of the ulnar MCP pivot, the first anchor washer may define an integrated lashing point formed by two openings to receive the second end of the palmar strap.

The backplate comprises a single MCP pivot that defines dual-sided anchor washers in a second aspect of the disclosed palmar strap assembly. In this embodiment, the dual-sided anchor washers define a radial attachment point for coupling with the first end of the palmar strap and an ulnar attachment point for coupling with the second end of the palmar strap.

These and other features, aspects, and advantages of the present disclosure will be better understood in the following description, appended claims, and accompanying drawings.

### BRIEF DESCRIPTION OF THE DRAWINGS

The drawing figures are not necessarily drawn to scale but to provide a better understanding of the components and are not intended to be limiting in scope but to provide exemplary illustrations. The figures illustrate exemplary configurations of a palmar strap assembly for a prosthetic device and in no way limit the structures or configurations according to the present disclosure. The non-limiting and non-exhaustive embodiments of the present invention, including the preferred embodiment, are described concerning the following figures, wherein reference numerals refer to like parts throughout the various views unless otherwise specified.

FIG. 1 is a perspective view of one embodiment of a palmar strap for a partial hand prosthesis.

FIG. 2 is a side perspective view of an embodiment of a partial hand prosthesis comprising a palmar strap.

FIG. 3A-3C illustrate an embodiment and components of an attachment anchor washer.

FIG. 3D illustrates an alternative embodiment of an attachment anchor washer.

FIG. 4A illustrates a side cross-sectional view of an MCP pivot joint and backplate.

FIG. 4B illustrates an exploded perspective view of the MCP pivot joint in FIG. 4A.

FIG. 5 illustrates a partial top view of an embodiment comprising a backplate and two variable-sized MCP pivot joints.

FIG. 6 illustrates a partial top view of a backplate comprising a single MCP pivot joint that defines a dual-sided anchor washer.

FIG. 7A illustrates a cross-sectional view of an alternative embodiment comprising an MCP pivot joint and backplate.

FIG. 7B illustrates exploded perspective views of the MCP pivot joint in FIG. 7A.

### DEFINITIONS

A description of a few terms is necessary for further ease of understanding the disclosure. As used herein, the term “proximal” has its ordinary meaning and refers to a location closer to the heart than another. Likewise, the term “distal” has its ordinary meaning and refers to a location that is further from the heart than another. The term “posterior” also has its ordinary meaning and refers to a location behind or the rear of another location. Lastly, the term “anterior” has its ordinary meaning and refers to a location that is ahead of or to the front of another location.

The term “palmar” refers to the palm (i.e., grasping side) of the hand. The palmar is also called the ventral side of the hand. The term “dorsal” means the opposite side of the hand, the back of your hand, or something that is towards the back of the hand. The term “radial” refers to the radius, the smaller bone in the forearm, or the side of the radius. The term “ulnar” refers to the ulna, a bone that spans from the elbow to the wrist on the medial side of the forearm when in anatomical position or the side of the ulna. Compared to the radius, the ulna is larger and longer.

The terms “rigid,” “flexible,” and “resilient” may be used herein to distinguish characteristics of portions of certain features of the prosthetic system. The term “rigid” denotes that an element of the system generally lacks flexibility. On the other hand, the term “flexible” is intended to denote that features are capable of repeated bending. The features may be bent into retained shapes or do not retain a general shape but continuously deform when force is applied. The term “resilient” qualifies as flexible features that return to an initial general shape without permanent deformation. The term “semi-rigid” connotes properties of elements that provide support and are free-standing; however, such elements may have some degree of flexibility or resiliency.

The term “anchor washer” or “palmar washer” refers to a washer-like element made of metal, rubber, or plastic that is secured in place by an articulation joint (e.g., screw) and serves as an attachment point for the palmar strap. The anchor washer may comprise multiple tabs to form an integrated lashing point or be constructed as a monolithic piece.

The term “attached” refers to elements being connected or united by fastening, adhering, bonding, etc., by any method suitable for the elements being fastened, secured, or joined together with their constituent materials. Many suitable methods for attaching elements are well-known, including adhesive bonding, pressure bonding, thermal bonding, mechanical fastening, etc. Such attachment methods may be used to attach elements over a particular area either continuously or intermittently. The term “attached” includes elements integrally formed with another element. The term “removably attached” refers to elements that may be connected or united, subsequently separated, and connected or united again by the same fastening means.

As used herein, the term “elastic” refers to any material which, upon application of a biasing force, is stretchable, that is, elongate, at least about 60 percent (i.e., to a stretched, biased length, which is at least about 160 percent of its relaxed unbiased length), and which, will recover at least 55 percent of its elongation upon release of the stretching, elongation force. Some clastic materials may be elongated by more than 60 percent (i.e., much more than 160 percent of their relaxed length), for example, by 100 percent or more. Many of these materials will recover to substantially their initial relaxed length, for example, to within 105 percent of their initial relaxed length, upon release of the stretch force. Such materials are referred to herein as “highly elastic.” As used herein, the term “nonelastic” refers to any material that does not fall within the definition of “elastic” (or “elastomeric”) or “highly elastic” above.

The term “hook fastening material” is used to designate a material having engaging elements designed to “hook” into complementary loop elements. The hook member may be referred to as a male fastener. It should also be understood that the use of the term “hook” should be non-limiting in the sense that the engaging elements may comprise any shapes as are known in the art so long as they are adapted to engage a complementary mechanical closure element such as a loop fastening material or another hook fastening material. The hook fastening material can mechanically engage fibrous elements of the corresponding loop member of the present disclosure to provide a secure closure. According to the present disclosure, a hook fastening material may be manufactured from a wide range of materials. Suitable materials include nylon, polyester, polypropylene, or any combination of these materials, or other materials as are known in the art.

The term “lashing point” refers to an attachment or fastening structure to which straps can be directly connected. Lashing points may include beckets, cleats, d-rings, O-rings, and equivalent structures.

The term “strap” refers to a long, narrow strip of material used for securing, holding together, or wrapping. The term “palmar strap” generally refers to a functionally continuous loop of a flexible textile material that wraps around the palm of a user for securing the backplate of a partial hand prosthesis (or prosthetic finger assembly) to the dorsal surface or backside of the hand of the user.

### DETAILED DESCRIPTION OF VARIOUS EMBODIMENTS

Various embodiments disclosed herein relate to a custom-designed, self-contained, bidirectional, biomechanically driven prosthetic finger assembly that can be fitted for a user with an amputated fingertip or finger segment. The streamlined and sophisticated design allows a patient with any residual finger to utilize a mechanical prosthetic finger that mimics a real finger's vertical and lateral motions and functionalities. The natural movement of the prosthetic finger assembly allows users to regain maximum control of the flexion, extension, abduction, and adduction movements of a fully functioning finger and fingertip. The assembly is designed to articulate in a realistic, natural manner in response to movement in the user's residual finger and/or adjacent fingers.

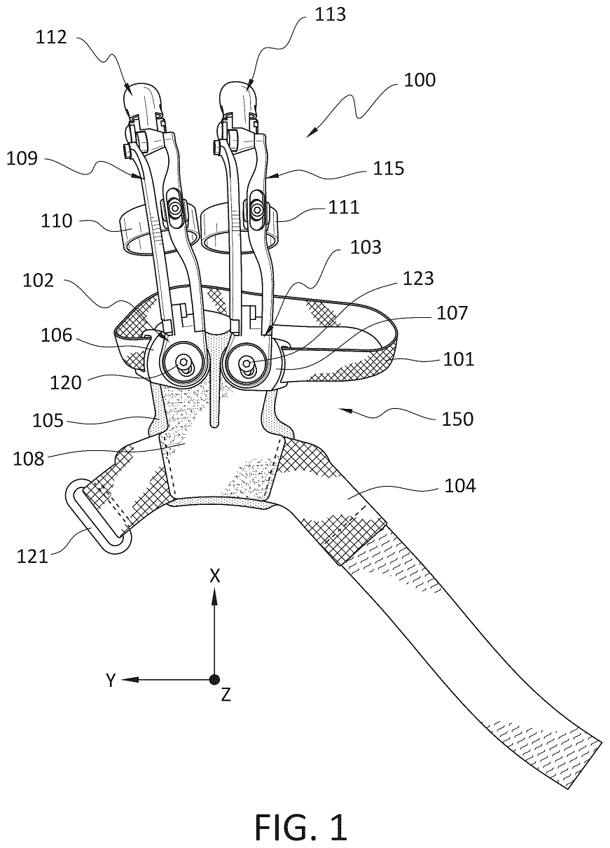

To facilitate an explanation of the movement of the bidirectional prosthetic finger discussed herein, relative vertical and lateral movements of the components of the prosthetic finger embodiments discussed below are explained concerning three axes—an x-axis, a y-axis, and a z-axis—initially defined in FIG. 1. These three axes inherently define two relevant planes of movement—an x-y plane and an x-z plane. Reference will now be made in detail to the accompanying drawings. The same reference numbers will be used throughout the drawings to refer to the same or like parts wherever possible. It is further understood that the use of relational terms such as first and second, top and bottom, and the like, if any, are used solely to distinguish one from another entity, item, or action without necessarily requiring or implying any actual such relationship or order between such entities, items or actions.

FIG. 1 illustrates a perspective view of one embodiment of a partial hand prosthesis or prosthetic finger assembly 100 comprising a palmar strap assembly 150 with a palmar strap 101. The partial hand prosthesis 100 generally refers to the entire device, including the palmar strap assembly 150, and the palmar strap assembly 150 generally refers to the various straps and fastening elements of the partial hand prosthesis 100. Unless otherwise specified, reference to the partial hand prosthesis 100 also includes reference to the palmar strap assembly 150. In this embodiment, partial hand prosthesis 100 includes a first or radial eccentric metacarpophalangeal (MCP) pivot 102 and a second or ulnar eccentric MCP pivot 103.

The radial MCP 102 pivot defines the most radial anchor position for the palmar strap 101. It is configured to attach to a user's hand on a backplate 108, which is adapted to attach to the back of a user's hand. The ulnar MCP pivot 103 defines the most ulnar anchor position for the palmar strap 101 and is also configured to attach to a user's hand on the backplate 108. The MCP pivots 102 and 103 each align with a different MCP joint and corresponding finger of the user. The backplate 108 may comprise a foam pad 105 mounted to a dorsal side of the backplate 108 and configured to receive the pivots 102 and 103. In an embodiment, the foam pad 105 is composed of silicone.

Embodiments of the partial hand prosthesis 100 may be formed of any suitable structural material that is non-irritating to human skin and allows the user to operate the device comfortably and confidently. Exemplary materials include titanium, stainless steel, aluminum, silicone, carbon fiber, nylon, plastic/polymer, wood, rubber, gold, silver, tungsten, flex cable, neoprene, or any other suitable material.

The partial hand prosthesis 100 comprises a first anchor (washer) 106 at the radial MCP 102 and a second anchor (washer) 107 at the ulnar MCP. The anchor washers 106 and 107, also called palmar washers, are adapted to receive the first and second ends of the palmar strap 101. The MCP pivots 102, 103 comprise articulation joints 120, 123 configured to rotate the MCP pivots 102, 103, respectively, relative to the hand of a user within a plane parallel to an x-y place and about an axis parallel to a z axis, the z-axis being defined in the ventral-dorsal directions. Articulation joints 120, 123 may be a pin, a screw, or any other appropriate fastener that joins anchor washers 106, 107 to the backplate 108 such that anchor washers 106, 107 revolve about axes parallel to the z-axis.

In an embodiment, the anchor washers 106 and 107 are mounted directly between the backplate 108 and MCP pivots 102, 103. The backplate 108 may vary in thickness, e.g., the backplate 108 may have an increased thickness at the most radial and ulnar anchor positions to provide an appropriate alignment and/or depth with the rest of partial hand prosthesis 100 and to avoid interference of the MCP pivots 102, 103 and anchor washers 106, 107 with the hand of a user.

In an embodiment, the palmar strap 101 of the partial hand prosthesis 100 attaches to the most radial anchor washer 106 and most ulnar anchor washer 107 mounted on a distal surface of the backplate 108. Alternatively, the palmar strap 101 may attach to the anchor washers 106, 107 being mounted on a distal surface of the backplate 108. In an embodiment, the first end of the palmar strap 101 is fixed to the first anchor washer 106.

The term “fixed” describes an element being sewn, welded, glued, or otherwise irremovably fastened. For example, the first end of the palmar strap 101 may be looped around the first anchor washer 106 and sewn to itself. In an embodiment, the first end of the palmar strap 101 comprises a rigid element (i.e., stud, crosspiece) that is enlarged to prevent the first end of the palmar strap 101 from separating from the first anchor washer 106. The second end of the palmar strap 101 is removably attached to the second anchor washer 107 to facilitate the donning and doffing of the device. The palmar strap 101 thus secures a distal end of the backplate 108 to the back of a user's hand to prevent backplate migration. In an embodiment, a second end of the palmar strap 101 is fixed to the second anchor washer 107, and the second end of the palmar strap 101 is removably attached to the first anchor washer 106. One skilled in the art can recognize the individualized needs of a wearer and may determine which anchor washer 106, 107 may be advantageously fixed for simplified donning and doffing of the partial hand prosthesis 100. In an alternative embodiment, the palmar strap 101 is removably attached to the first anchor washer 106 and the second anchor washer 107. Such an embodiment allows the palmar strap 101 to be easily replaced and cleaned. The palmar strap 101 can be tensioned simultaneously on both sides of the backplate 108 or adjusted individually according to the user's demands. In an embodiment, the palmar strap 101 is composed of clastic material conforming to the shape of the user's hand. In such an embodiment, the palmar strap 101 may be fixed or adjustable in length.

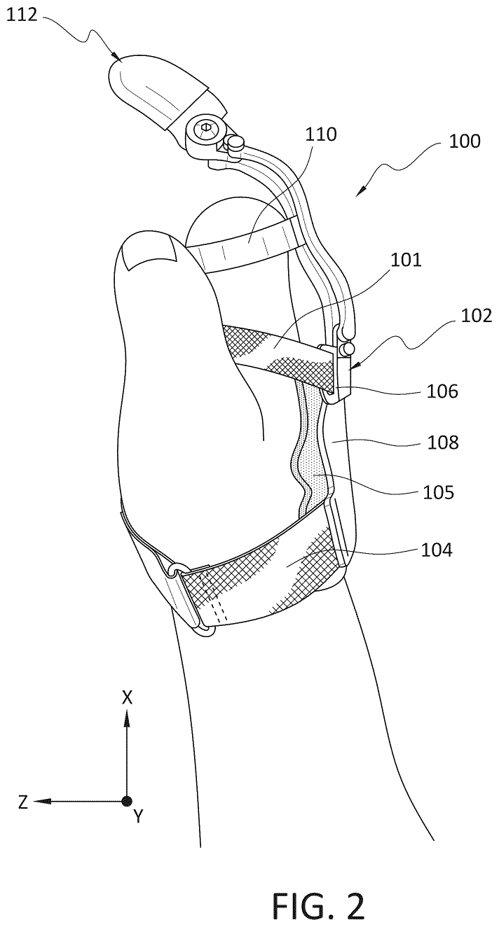

FIG. 2 illustrates a side perspective view of the partial hand prosthesis 100. The palmar strap 101 is preferably formed from a textile-based material but can be constructed from various materials such as polymers and combinations of polymers and textiles. The palmar strap 101 may have at least one surface formed from hook receivable material and/or hook engaging material. The palmar strap 101 is preferably constructed of an elastic or non-elastic material to wrap around a user's palm. In an embodiment, the palmar strap 101 is made from nylon, polyamide, polyester, polypropylene, elastane, neoprene, foam, and/or another flexible textile. It should be understood, however, that several of the embodiments disclosed herein may be equally applicable to various types of strap material, and not just to the flat, two-sided material shown in the figures. In an embodiment, the palmar strap 101 is trimmable to customize the straps' length. In an embodiment, the palmar strap assembly 150 may utilize a dial tensioning device at one or more MCP pivot 102, 103, joints to secure a palmar strap 101 about a user's hand.

The palmar strap 101 is preferably connected to the anchor washers 106, 107 to allow for convenient positioning of the palmar strap 101 about the hand and to avoid uncomfortable interference with the dorsal surface of the hand. In an alternative embodiment, the palmar strap 101 is adapted to attach directly to the backplate 108, wherein the backplate 108 comprises one or more integrated slots or openings as anchors to receive the ends of the palmar strap 101 at a thickened, distal end of the backplate 108. In an embodiment, the first end of the palmar strap 101 comprises a rigid element (i.e., stud, crosspiece) that is enlarged to prevent the first end of the palmar strap 101 from separating from the first anchor washer 106. The second end of the palmar strap 101 comprises a hook and an attachment mechanism (e.g., a high-density hook-and-loop attachment mechanism, such as is commonly sold under the brand name Velcro™). The second end of the palmar strap 101 may be devoid of an attachment mechanism in embodiments wherein the second end is configured to fasten to the backplate 108 or anchor washer 107 at a lashing point 117.

The partial hand prosthesis 100 preferably comprises a wrist strap 104 adapted to wrap around a user's wrist and secure a proximal portion of the partial hand prosthesis 100 to the back of a user's hand. In an embodiment, the wrist strap 104 comprises a first end that is anchored to a proximal, ulnar side of the backplate 108 and a second end that is configured to secure to a fastening means 121 (i.e., D-ring, hook, and loop textile material, magnetic clasp, button, clasp, clip, dial tensioning device, ratchet, buckle). One skilled in the art will recognize that the wrist strap 104 may instead comprise a first end that is anchored to a proximal, radial side of the backplate 108 and a second end that is configured to be secured to a fastening means, depending on the individual needs of a wearer of the partial hand prosthesis 100. The wrist strap 104 is preferably constructed of an elastic or non-elastic material to wrap around a user's wrist. In an embodiment, the wrist strap 104 is made from nylon, polyamide, polyester, polypropylene, elastane, neoprene, foam, and/or another flexible textile. It should be understood, however, that several of the embodiments disclosed herein may be equally applicable to various types of strap material and not just to the flat, two-sided material shown in the figures.

According to a variation, the MCP pivots 102, 103 comprise prosthetic fingers 109, 115, respectively, as disclosed in U.S. Pat. No. 9,707,103, the disclosure of which is incorporated by reference. For example, as described in greater detail in U.S. Pat. No. 9,707,103, the MCP pivots 102, 103 may also include a frame, articulation assembly, distal coupler, proximal rocker, hinged connection, adjustable ring tendon, and the like. The embodiment depicted in FIG. 1 illustrates the radial MCP pivot 102 as being connected to a first prosthetic finger 109 comprising a first ring 110 and a first distal coupler 112, and the ulnar MCP pivot 103 as being connected to a second prosthetic finger 115 comprising a second ring 111 and a second distal coupler 113.

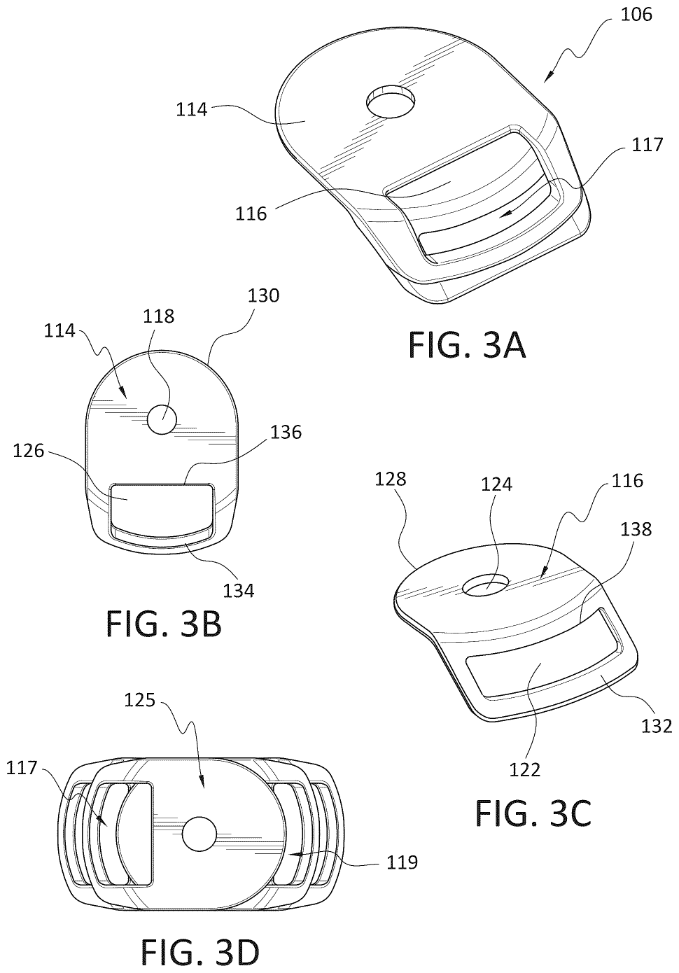

FIG. 3A depicts an exemplary embodiment of an anchor washer 106 of the present disclosure. Embodiments of the anchor washer 106 may also be applied to the anchor washer 107, depending on the application of the radial MCP pivot 102 and ulnar MCP pivot 103. The anchor washer 106 preferably comprises an integrated lashing point 119 formed by a base tab 116 and an upper tab 114. The base tab 116 is directly mounted to the backplate 108, and the upper tab 114 is mounted directly to the base tab 116. Thus, the base tab 116 and the upper tab 114 for a unitary anchor washer 106 are united together to form a coordinate entity so that they do not require separate manipulative parts. The anchor washer 106 is adapted to pivot about an axis defined by the radial MCP pivot 102 in the x-y plane and is positionable to allow the palmar strap 101 to conform to the shape of the wearer's hand functionally. By providing the anchor washer 106 with an integrated lashing point 119, the palmar strap 101 can be advantageously devoid of bulky fasteners (e.g., D-rings) that may directly interfere or uncomfortably engage with the palmar surface, ulnar border, and radial border of a user's hand.

FIG. 3B illustrates an embodiment of an upper tab 114 of the anchor washer 106. The upper tab 114 preferably comprises a first aperture 118 in the main plate 130 of the upper tab 114 to receive an articulation joint 120. The main plate 130 is aligned in the x-y plane and rotates about an axis parallel to the z-axis. The upper tab 114 comprises a slot or an opening 126 to receive the palmar strap 101 and defines an upper rod 134 and a flat edge 136 that form part of the lashing point 119.

FIG. 3C illustrates an embodiment of a base tab 116 of the anchor washer 106. The base tab 116 preferably comprises a second aperture 124 in the main plate 128 of the base tab 116 to receive an articulation joint 120. The main plate 128 is aligned in the x-y plane and rotates about an axis parallel to the z-axis. The base tab 116 comprises an arcuate slot or opening 122 to receive the palmar strap 101 and defines a base rod 132 and an arcuate edge 138 that form part of the lashing point 119. The palmar strap 101 is adapted to attach to the lashing point 119 by going up through openings 122, 126, wrapping about the upper rod 134, and returning through the base opening 122. One skilled in the art will recognize that other configurations of the lashing point 119 (i.e., alternative fastening means) may be advantageously used depending on the application.

FIG. 3D illustrates an exemplary embodiment wherein the anchor washer 125 is dual-sided, comprising a first or radial lashing point 117 and a second or ulnar lashing point 119. The dual-sided anchor washer 125 may be used for a single prosthetic finger in partial hand prosthesis 100 applications. The dual-sided anchor washer 125 simplifies the design for applications of single-finger prosthetic devices by providing a single, rotatable attachment point or pivot instead of two (e.g., radial and ulnar) attachment points or pivots. In an alternative embodiment, lashing point 117 comprises one slot or opening, and 119 comprises two slots or openings. The lashing points 117, 119 of the dual-sided anchor washer 125 may be identical or different.

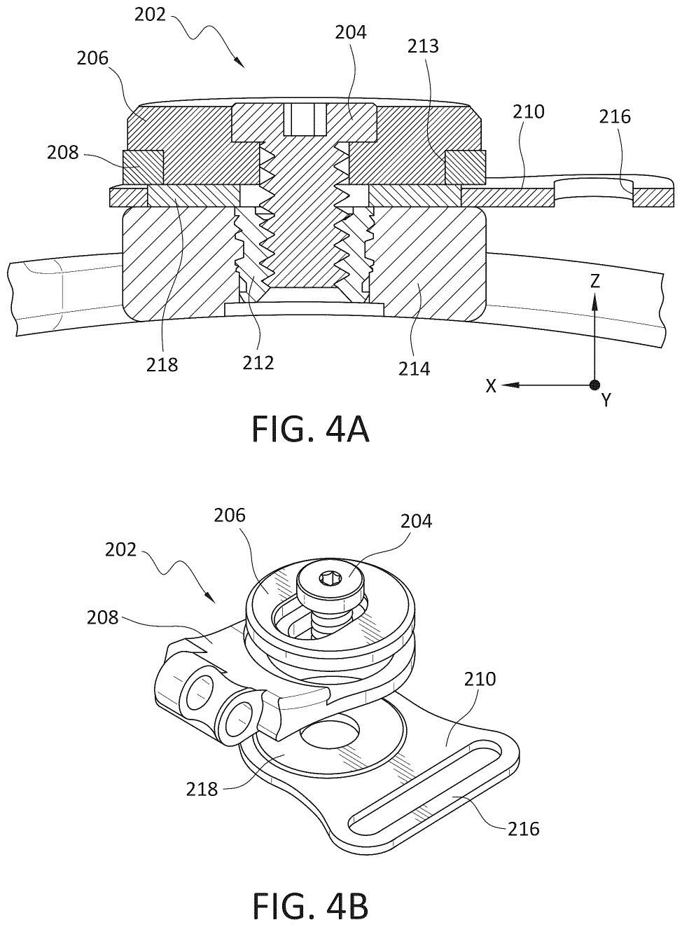

FIGS. 4A and 4B illustrate side and exploded views, respectively, of an MCP pivot 202 joint provided with a cap washer 206 having a circular hole to retain an articulation joint 204, wherein the cap washer 206 remains stationary if an MCP frame 208 or palmar washer 210 is offset. The MCP pivot 202 joint is mounted on the backplate 214. The frame 208 is attached to the backplate 214 by means of the articulation joint 204 (e.g., screw) and an anchor piece 212 (e.g., nut). In an embodiment, the articulation joint 204 is a titanium screw. The articulation joint 204 may be directly integrated or fastened into a thickness of the backplate 214. The cap washer 206 surrounds the articulation joint 204 above the backplate 214, and the washer is fixed on the backplate 214 and above the frame 208. In an embodiment, the washer may be a Delrin washer composed of a high-performance acetal resin (e.g., polyoxymethylene (POM)).

The frame 208 is rotatably fixed about a groove 213 of the washer and is configured to rotate about the articulation joint 204. The palmar washer 210, also referred to as an anchor washer (e.g., anchor washer 106), comprises a lashing point 216 to attach to a palmar strap. The palmar washer 210 is rotatably fastened between the frame 208 and backplate 214, wherein a spacer 218 is disposed between the cap washer 206 and backplate 214. The spacer 218 is planar with the palmar washer 210. In an embodiment, the spacer 218 is composed of an elastic material and configured to distribute pressure between the cap washer 206, frame 208, and backplate 214.

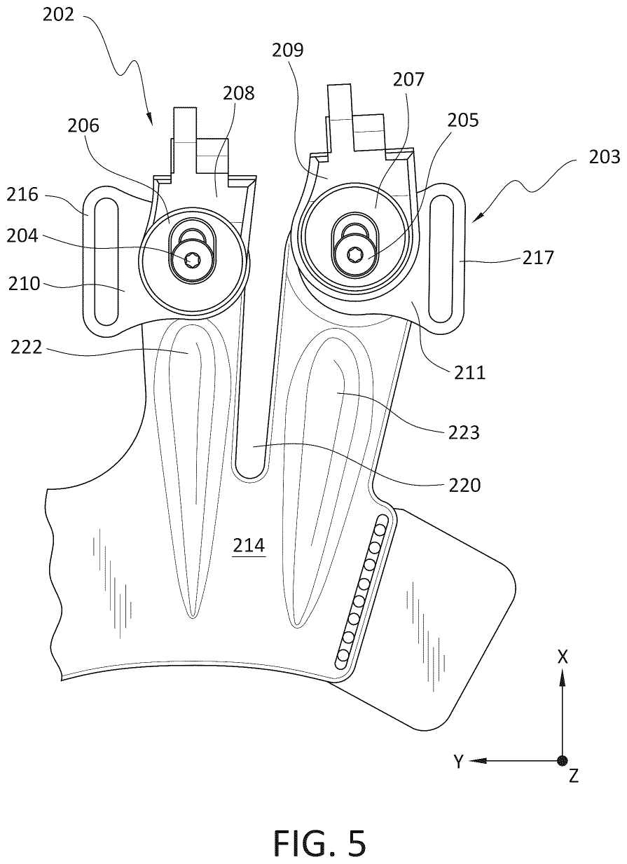

FIG. 5 illustrates a partial top view of a backplate 214 with a first or radial MCP pivot 202 joint, as observed in FIG. 4B, and a second or ulnar MCP pivot 203 joint. The second MCP pivot 203 joint is depicted as smaller than the first MCP pivot 202 to accommodate sizing and various anatomies of a user. In an embodiment, the first MCP pivot 202 is mounted to radial distal projection 222 of the backplate 214. The second MCP pivot 203 is mounted to an ulnar distal projection 223 of the backplate 214. The backplate 214 forms a gap 220 between the radial distal projection 222 and the ulnar distal projection 223.

Depending on the application (i.e., users with various prosthetic finger requirements), other distal projections and MCP pivots may be disposed between the most radial distal projection 222 and most ulnar projection 223. The frames 208, 209 are configured to attach prosthetic fingers (e.g., prosthetic finger 109). In an embodiment, the second MCP pivot 203 has a mirrored configuration to the first MCP pivot 202. The second MCP pivot 203 is provided with a cap washer 207 having a circular hole to retain an articulation joint 205, wherein the cap washer 207 remains stationary if an MCP frame 209 or palmar washer 211 is offset. The second MCP pivot 203 joint is mounted on the backplate 214. The frame 209 is attached to the backplate by means of the articulation joint 205. The cap washer 207 surrounds the articulation joint 205 above the backplate 214, and the washer is fixed on the backplate 214 and above the frame 209. The palmar washer 211 comprises a lashing point 217 to attach to a palmar strap. The palmar washer 211 is rotatably fastened between the frame 209 and backplate 214.

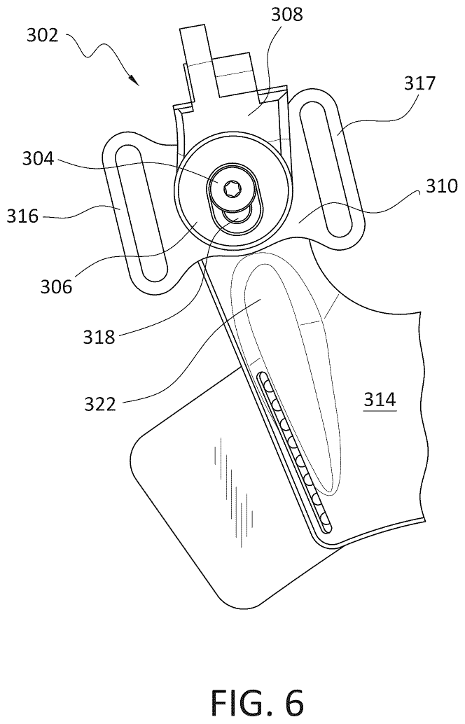

FIG. 6 illustrates a partial top view of an alternative backplate 314 with an MCP pivot 302 having a dual-sided attachment configuration. The MCP pivot 302 features a configuration similar to the MCP pivot 202 configuration in FIGS. 4A-4B. The MCP pivot 302 joint is provided with a cap washer 306 having a circular hole to retain an articulation joint 304, wherein the cap washer 306 remains stationary if an MCP frame 308 or palmar washer 310 is offset. A spacer 318 composed of flexible material (e.g., rubber) is disposed of between the cap washer 306 and backplate 314.

The MCP pivot 302 is mounted on a distal projection 322 arranged for a partial hand prosthesis having a single prosthetic finger. The frame 308 is arranged to connect with a prosthetic finger (e.g., prosthetic finger 109). The palmar washer 310 comprises a radial lashing point 316 and an ulnar lasing point 317 for securing a palmar strap about a user's hand. The palmar washer 310 is rotatably adjustable about the articulation joint 304.

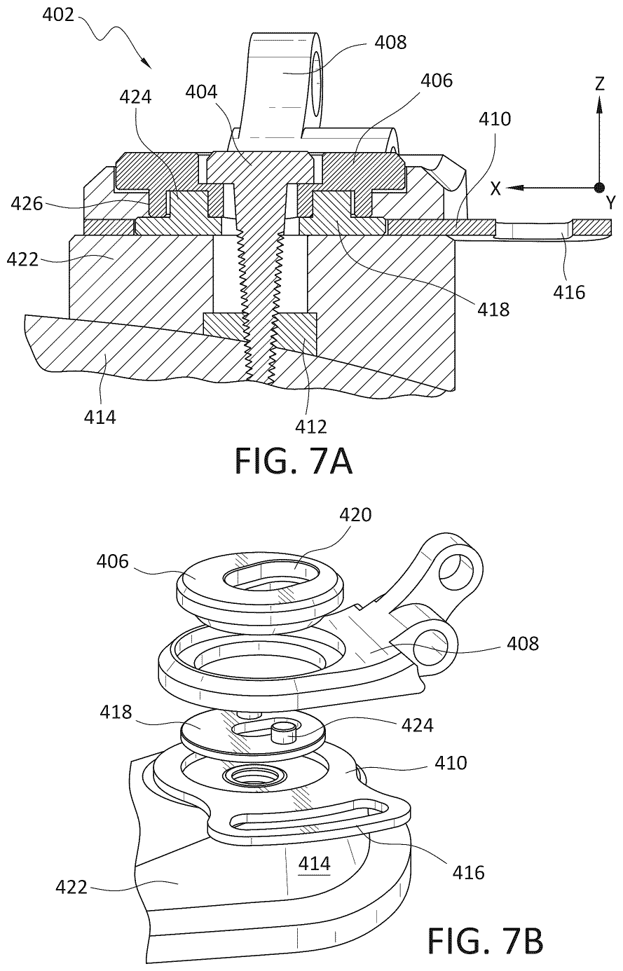

FIGS. 7A and 7B illustrate side and exploded views, respectively, of an MCP pivot 402 joint provided with an acetal spacer 418 that mates with the geometry of the cap washer 406 with a slotted aperture 420 to retain the screw. The spacer 418 moves with the cap washer 406 if it is offset in the x or y directions. The MCP pivot 402 joint is mounted on the backplate 414. The frame 408 is attached to the backplate 414 utilizing the articulation joint 404 (e.g., screw) and an anchor piece 412 (e.g., nut).

The articulation joint 404 may be mounted on a distal projection 422 of the backplate 414 and directly integrated or fastened into a thickness of the backplate 414. The cap washer 406 surrounds the articulation joint 404 above the backplate 414. The cap washer 406 forms at least one mortise 426 to receive at least one tenon 424 of the spacer 418. The palmar washer 410, also referred to as an anchor washer (e.g., anchor washer 106), comprises a lashing point 416 to attach to a palmar strap. The palmar washer 410 is rotatably fastened about the spacer 418 and between both the frame 408 and backplate 414, wherein the spacer 418 is disposed between the cap washer 406 and backplate 414. In an embodiment, the spacer 418 is composed of an elastic material and configured to distribute pressure between the cap washer 406, frame 408, and backplate 414.

It is understood that not all objects or advantages may be achieved under any embodiment of the disclosure. Those skilled in the art will recognize that a palmar strap assembly for a partial hand prosthesis may be embodied or carried out to achieve or optimize one advantage or group of advantages as taught herein without achieving other objects or advantages as taught or suggested herein.

The skilled artisan will recognize the interchangeability of various disclosed features. Besides the variations described herein, other known equivalents for each feature can be mixed and matched by one of ordinary skill in this art to build and use a palmar strap assembly under the principles of the present disclosure. The skilled artisan will understand that the features described herein may be adapted to other methods and types of prosthetic devices/applications.

It is intended that the present disclosure should not be limited by the disclosed embodiments described above and may be extended to other applications that may employ the features described herein.

1. A strap assembly comprising:
a backplate including a radial metacarpophalangeal (MCP) pivot and an ulnar MCP pivot, the radial MCP pivot defining a first anchor, and the ulnar MCP defining a second anchor; and
a palmar strap defining a first end and a second end;
wherein the first anchor defines a first attachment point for engaging with the first end of the palmar strap, and the second anchor defines a second attachment point for engaging with the second end of the palmar strap.

2. The strap assembly of claim 1, wherein the first anchor is positionable about a radial MCP axis, and the second anchor is positionable about an ulnar MCP axis, the radial MCP and ulnar MCP axes are defined in ventral-dorsal directions.

3. The strap assembly of claim 2, wherein the first anchor includes a base tab and an upper tab.

4. The strap assembly of claim 3, wherein the base tab comprises:
a first aperture corresponding to the radial MCP axis and arranged to receive a first articulation joint; and
an arcuate slot configured and dimensioned to receive the first end of the palmar strap.

5. The strap assembly of claim 4, wherein the upper tab comprises:
a second aperture corresponding to the radial MCP axis, and configured and dimensioned to receive the first articulation joint; and
an opening adapted to receive the first end of the palmar strap, the opening corresponding to and aligned with the arcuate slot of the base tab.

6. The strap assembly of claim 5, wherein the upper tab and the base tab are mounted to the backplate by the first articulation joint.

7. The strap assembly of claim 1, further comprising a wrist strap defining a first end and a second end, wherein the first end is integrally secured to a proximal, ulnar end of the backplate.

8. The strap assembly of claim 1, wherein the first end of the palmar strap is fixed to the first attachment point of the first anchor, and the second end of the palmar strap is removably attached to the second attachment point of the second anchor.

9. The strap assembly of claim 1, wherein the second end of the palmar strap is fixed to the second attachment point of the second anchor, and the first end of the palmar strap is removably attached to the first attachment point of the first anchor.

10. A prosthetic finger assembly comprising:
a backplate including a radial metacarpophalangeal (MCP) pivot and an ulnar MCP pivot, the radial MCP pivot defining a first anchor and the ulnar MCP defining a second anchor;
a first articulation assembly rotatively coupled between the radial MCP pivot and a first distal coupler;
a second articulation assembly rotatively coupled between the ulnar MCP pivot and a second distal coupler; and
a palmar strap defining a first end and a second end, the second end being removably attached to the second anchor of the ulnar MCP pivot;
wherein the first anchor is positionable about a radial MCP axis and the second anchor is positionable about an ulnar MCP axis, the radial MCP and ulnar MCP axes being defined in ventral-dorsal directions.

11. The prosthetic finger assembly of claim 10, wherein the first end is fixed to the first anchor of the radial MCP pivot.

12. The prosthetic finger assembly of claim 10, wherein the second anchor defines an integrated lashing point formed by a first opening and a second opening to receive the second end of the palmar strap.

13. The prosthetic finger assembly of claim 12, wherein the first opening is formed by a base tab and the second opening is formed by an upper tab.

14. The prosthetic finger assembly of claim 10, wherein the second anchor defines a receiving ring arranged to receive the second end of the palmar strap.

15. The prosthetic finger assembly of claim 10, wherein the second anchor defines a textile tab arranged to engage with the second end of the palmar strap.

16. The prosthetic finger assembly of claim 15, wherein the textile tab comprises hook fastener material and the second end of the palmar strap comprises loop fastener material.

17. A prosthetic finger assembly comprising:
a backplate having a radial metacarpophalangeal (MCP) pivot and an ulnar MCP pivot, the radial MCP pivot defining a first anchor and the ulnar MCP defining a second anchor;
a first articulation assembly rotatively coupled between the radial MCP pivot and a first distal coupler;
a second articulation assembly rotatively coupled between the ulnar MCP pivot and a second distal coupler; and
a palmar strap defining a first end and a second end, the first end being removably attached to the first anchor of the radial MCP pivot;
wherein the first anchor is positionable about a radial MCP axis and the second anchor is positionable about an ulnar MCP axis, the radial MCP and ulnar MCP axes being defined in ventral-dorsal directions.

18. The prosthetic finger assembly of claim 17, wherein the second end is fixed to the second anchor of the ulnar MCP pivot.

19. The prosthetic finger assembly of claim 17, wherein the first anchor defines an integrated lashing point formed by first and second openings to receive the second end of the palmar strap.

20. The prosthetic finger assembly of claim 19, wherein the first opening is formed by a base tab and the second opening is formed by an upper tab.
